(19) United States
(12) Patent Application Publication
(10) Pub. No.: US 2011/0126370 A1
(43) Pub. Date: Jun. 2, 2011

(54) Self Sanitizing Electronic Toothbrush Apparatus

(76) Inventor: David Isidore Reuben, Las Vegas, NV (US)

(21) Appl. No.: 13/023,665

(22) Filed: Feb. 9, 2011

Related U.S. Application Data
(63) Continuation of application No. 11/426,066, filed on Jun. 23, 2006.

(51) Int. Cl. A46B 9/04; A46B 17/06

(52) U.S. Cl. 15/167.1; 250/455.11

(57) ABSTRACT

A self sanitizing toothbrush comprising a toothbrush handle and an interactive fitted electronic cap which sanitizes the toothbrush head automatically after brushing.

### CROSS REFERENCE TO RELATED APPLICATIONS

This is a continuation of application # 11426066 filed Jun. 23, 2006.

### FIGURES

FIG. 1 Toothbrush Front

FIG. 2 Toothbrush Side

FIG. 3 Toothbrush Side Alternate Embodiment

FIG. 4 Toothbrush Handle

FIG. 5 Cap Side

FIG. 6 Cap Bottom

FIG. 7 Toothbrush Back

FIG. 8 Expanded View of FIG. 2

FIG. 9 Toothbrush Side With Spring Ball Attachment

FIG. 10 Three Dimensional Non Line Drawing Showing Toothbrush Structure

### REFERENCE NUMERALS

10 Light Emitting Element
11 Battery
12 Micro Reed Switch
13 Digital Timer
14 Magnet
15 Colored LED
16 Fiber Optic Indicator
17 Fiber Optic Strands
18 Latch
19 Indicator LED
20 Spring Ball

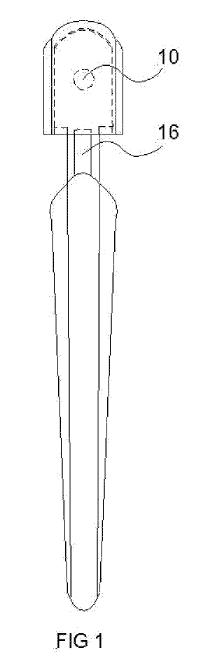

### DETAILED DESCRIPTION—FIGS 1-10

FIG. 1 shows the front of the toothbrush. The sanitizing light emitting element 10 and associated electronics are hidden within the cap. All that is visible from the front is the fiber optic indicator 16.

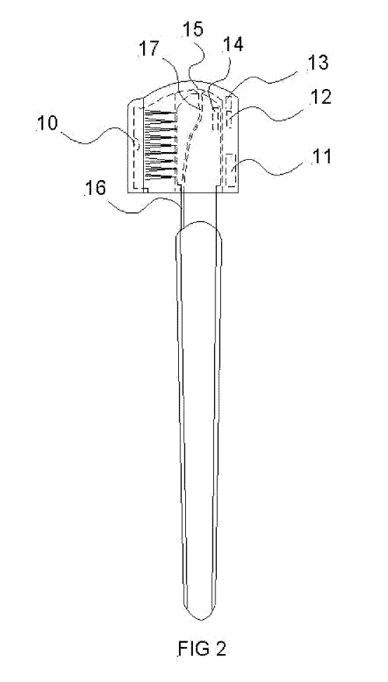

FIG. 2 shows the side of the toothbrush. The sanitizing light emitting element 10 is shown in the front of the cap and is oriented to irradiate the bristles. The battery 11 is shown sealed in the back of the cap. Control electronics including micro reed switch 12 and digital timer 13 are also shown sealed in the back of the cap. The magnet 14 which activates the micro reed switch 12 is shown embedded in the toothbrush handle. Fiber optic indicator 16 is shown in the front. The fiber optic strands 17 which illuminate the indicator 16 are shown embedded in the toothbrush handle. These strands are illuminated by the colored LED 15 shown at the top of the inside of the cap.

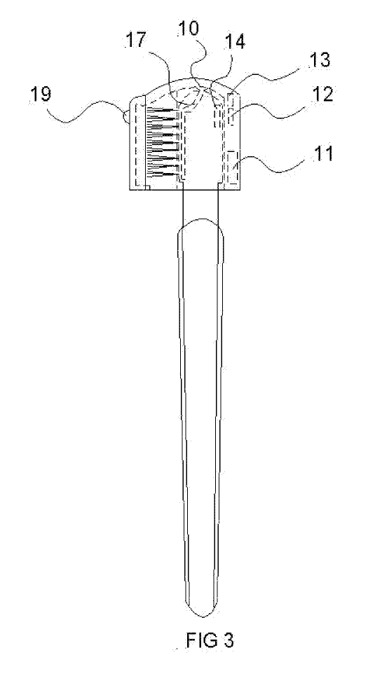

FIG. 3 shows the side of the toothbrush. In this embodiment the sanitizing light emitting element 10 is shown at the top of the inside of the cap. In this embodiment the sanitizing light emitting element 10 illuminates the fiber optic strands 17 which run behind the bristles and sanitize them from below. In this embodiment the fiber optic strands 17 are side emitting and in one embodiment are capable of transmitting deep UV. In lieu of a fiber optic indicator an indicator LED 19 is used and is shown on the outside of the front of the cap. Placement of the battery 11 and electronic components 12 and 13 are shown the same as in FIG. 2.

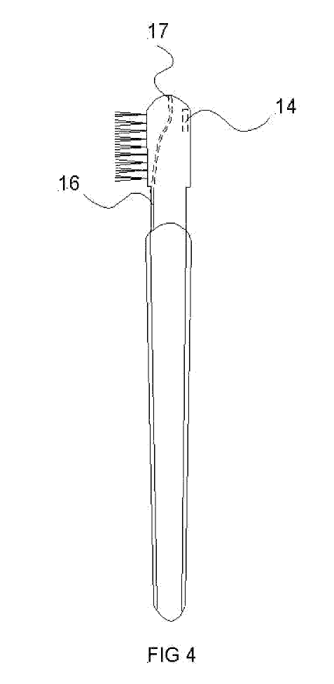

FIG. 4 shows the side of the toothbrush handle. The fiber optic strands 17 which transmit light from the colored LED 15 to the fiber optic indicator 16 are shown embedded in the head. Also shown embedded in the head is the magnet 14 which activates the micro reed switch 12 in the cap.

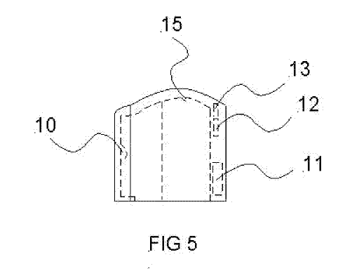

FIG. 5 shows a side view of the cap in FIG. 2. All electronic components including the sanitizing light emitting element 10 colored LED 15 digital timer 13 micro reed switch 12 and battery 11 are shown sealed inside the cap.

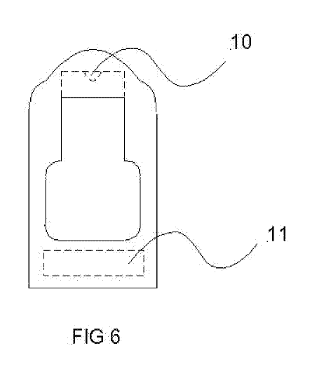

FIG. 6 shows a bottom view of the cap. The sanitizing light emitting element 10 is shown in the front of the inside of the cap. The battery 11 is shown sealed in the back of the cap. The toothbrush handle shown in FIG. 4 slips into the hole shown.

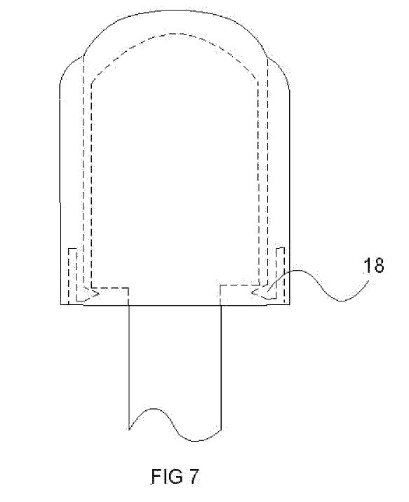

FIG. 7 shows the back of the toothbrush sans the electronics. Latch 18 is shown inside the cap. This latch latches the cap onto the toothbrush handle.

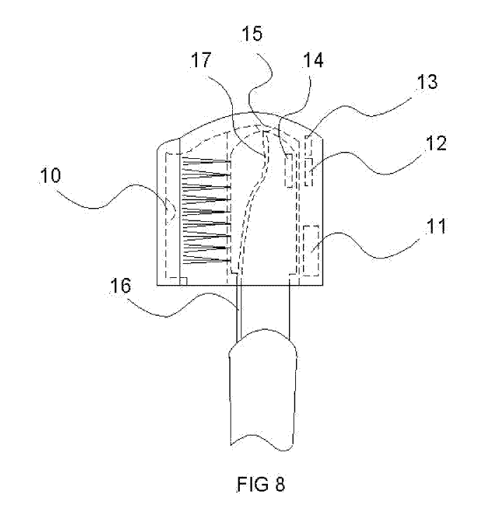

FIG. 8 shows an expanded view of FIG. 2.

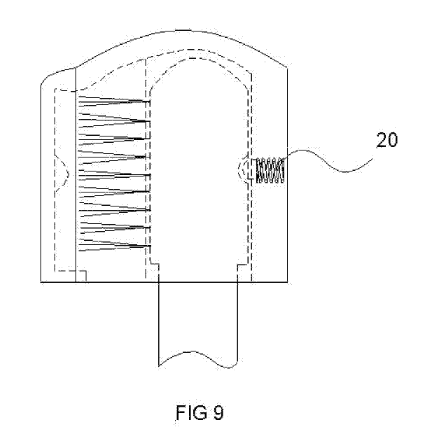

FIG. 9 shows the location of a spring ball 20 inside the cap. The spring ball can be used to attach the cap to the toothbrush head via an indentation shown in the back of the toothbrush head. A spring ball switch in the same location can be used for activating the digital timer 13.

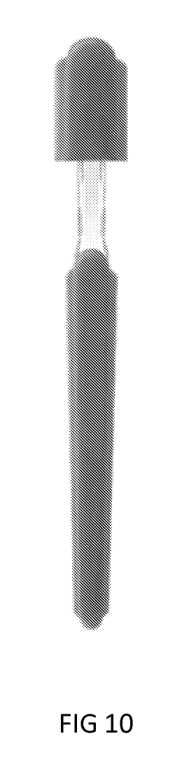

FIG. 10 shows a three dimensional non line drawing embodiment of the toothbrush showing the toothbrush structure.

### DETAILED DESCRIPTION—OPERATION

In the preferred embodiment of this product the consumer would purchase the product as they would an ordinary toothbrush. The toothbrush would come prepackaged with the cap similar to the embodiment shown in FIG. 10. The toothbrush would have an advertised lifetime of, for example, between two and three months and it would operate, for example, for 150 sanitizing cycles.

The consumer would brush their teeth normally and then after brushing slip the cap onto the toothbrush head. The cap is fitted to the toothbrush head and attaches itself to it. The fiber optic indicator would then flash indicating a sanitizing cycle has begun. This indicator is also the indicator for the integrity of the battery charge. In the alternate embodiment an LED on the outside of the cap takes the place of the fiber optic indicator. The consumer can then put the toothbrush away while it sanitizes itself.

Once the indicator stops flashing when the cap is slipped onto the toothbrush the consumer would know that the product has expired and can then dispose of the toothbrush or send it in for recycling and purchase a new toothbrush.

The American Dental Association recommends that consumers replace their toothbrush every two to three months. This product compels the consumer to do so. Since the product has a finite lifetime, contamination issues associated with long term use are avoided.

Several sanitization technologies can be employed in this device. These technologies include:

1. Deep UV LED Technology: LED chips emitting at or near the peak germicidal frequency, 265 nm, are commercially available and would be ideal for use in this product.

2. UVA LEDs+TiO2: The threshold wavelength for TiO2 is 388 nm so UVA LEDs emitting below that in combination with the titanium dioxide photocatalyst can be used in this product.

3. HINS Light: HINS light, 405 nm, has been shown to be effective against staph bacteria and MRSA. LEDs emitting at that wavelength can be used in this product.

4. Miniature Mercury Vapor Bulbs: Miniature mercury vapor bulbs are commercially available and can be used in this product.

### DETAILED DESCRIPTION—OBJECTS AND OBJECTIVES

Aside from being a different structure (toothbrush, FIG. 10) the proposed device offers several critical advantages over conventional universal toothbrush sanitizers. These advantages include:

1. The emitter is positioned perfectly in front of the target area (bristles) each time the device is used.

2. Contamination due to long term use is avoided.

3. Consumers are compelled to exchange their toothbrush at the recommended period (2-3 months).

4. The device is portable.

I claim:

1. A toothbrush having a congruous fitted interactive cap that embodies one or more light emitting elements which automatically irradiate the head of the toothbrush and sanitize it after use.

2. The toothbrush in claim 1 wherein the light emitting elements in the cap are LEDs.

3. The toothbrush in claim 1 wherein the light emitting element in the cap is a bulb.

4. The toothbrush in claim 1 wherein the light is directly actinic.

5. The toothbrush in claim 1 wherein the light activates a photocatalyst.

6. The LEDs in claim 2 wherein the light emitted is of ultraviolet wavelength.

7. The LEDs in claim 2 wherein the light emitted is HINS light.

8. The ultraviolet wavelength in claim 6 wherein the said wavelength is at or near the resonant frequency of DNA for microorganisms.

9. The toothbrush in claim 1 wherein irradiation is controlled by a digital timer embedded in the cap.

10. The toothbrush in claim 1 wherein irradiation is digitally pulsed.

11. The timer in claim 9 wherein the timer is activated by a reed switch in the cap.

12. The timer in claim 9 wherein the timer is activated by a spring ball switch in the cap.

13. The toothbrush in claim 1 wherein the handle of the toothbrush contains embedded magnetic triggering material.

14. The toothbrush in claim 1 wherein the battery is embedded in the cap.

15. The battery in claim 14 wherein the battery is rechargeable.

16. The battery in claim 14 wherein the battery is replaceable.

17. The toothbrush in claim 1 wherein the head of the toothbrush is coated with titanium dioxide photocatalyst.

18. The toothbrush in claim 1 wherein the bristles are made of a titanium dioxide composite polymer.

19. The toothbrush in claim 1 wherein the inside of the cap is coated with reflective material.

20. The toothbrush in claim 1 wherein the toothbrush handle contains fiber optic strands which are illuminated by an LED in the cap.

21. The toothbrush in claim 1 where in the cap is slip fit onto the toothbrush head.

22. The toothbrush in claim 1 where in the cap is latched onto the toothbrush head.

23. The toothbrush in claim 1 wherein the cap is attached to the toothbrush head via a spring ball.

24. The toothbrush in claim 1 wherein the lifetime of the battery in the cap is calibrated to match the expected lifetime of the bristles so that the entire toothbrush is disposed of as a unit at the end of the said lifetime.

25. The toothbrush in claim 1 wherein the toothbrush is recyclable.
